SEQUENCE LISTING (3 sequences)

SEQ ID NO: 1 - DNA, 20 bp, Artificial Sequence
  Forward primer 27F for bacterial 16S rDNA
agagtttgatcmtggctcag

SEQ ID NO: 2 - DNA, 20 bp, Artificial Sequence
  Reverse primer pH′ for bacterial 16S rDNA
aaggaggtgatccagccgca

SEQ ID NO: 3 - DNA, 1546 bp, Bacillus coagulans strain RBE4-4
agagtttgatcatggctcaggacgaacgctggcggcgtgcctaatacatgcaagtcgtgcggaccttttaaaagcttgcttttaaaaggttagcggcggacgggtgagtaacacgtgggcaacctgcctgtaagatcgggataacgccgggaaaccggggctaataccggatagttttttcctccgcatggaggaaaaaggaaagacggcttcggctgtcacttacagatgggcccgcggcgcattagcttgttggtggggtaacggctcaccaaggcaacgatgcgtagccgacctgagagggtgatcggccacattgggactgagacacggcccaaactcctacgggaggcagcagtagggaatcttccgcaatggacgaaagtctgacggagcaacgccgcgtgagtgaagaaggccttcgggtcgtaaaactctgttgccggggaagaacaagtgccgttcgaacagggcggcgccttgacggtacccggccagaaagccacggctaactacgtgccagcagccgcggtaatacgtaggtggcaagcgttgtccggaattattgggcgtaaagcgcgcgcaggcggcttcttaagtctgatgtgaaatcttgcggctcaaccgcaagcggtcattggaaactgggaggcttgagtgcagaagaggagagtggaattccacgtgtagcggtgaaatgcgtagagatgtggaggaacaccagtggcgaaggcggctctctggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacctccctggagacagggccttccccttcgggggacagagtgacaggtggtgcatggttgtcgtcagctcgtgtcgtgagatgttgggttaagtcccgcaacgagcgcaacccttgaccttagttgccagcattcagttgggcactctaaggtgactgccggtgacaaaccggaggaaggtggggatgacgtcaaatcatcatgccccttatgacctgggctacacacgtgctacaatggatggtacaaagggctgcgagaccgcgaggttaagccaatcccagaaaaccattcccagttcggattgcaggctgcaacccgcctgcatgaagccggaatcgctagtaatcgcggatcagcatgccgcggtgaatacgttcccgggccttgtacacaccgcccgtcacaccacgagagtttgtaacacccgaagtcggtgaggtaacctttacggagccagccgccgaaggtgggacagatgattggggtgaagtcgtaacaaggtagccgtatcggaaggtgcggctggatcacctcctt

(19) United States
(12) Patent Application Publication
(10) Pub. No.: US 2020/0002735 A1
(43) Pub. Date: Jan. 2, 2020

(54) LACTIC ACID-PRODUCING BACILLUS COAGULANS STRAIN AND USE THEREOF

(71) Applicant: FAR EASTERN NEW CENTURY CORPORATION, Taipei City (TW)

(72) Inventors: Yu-Chuan CHUANG, Taipei City (TW); To-Chun CHAO, Taipei City (TW)

(21) Appl. No.: 16/387,948

(22) Filed: Apr. 18, 2019

(30) Foreign Application Priority Data
Jun. 29, 2018 (TW) 107122573

(51) Int. Cl. C12P 7/56; C12N 1/20

(52) U.S. Cl. CPC C12P 7/56; C12N 1/20

(57) ABSTRACT

Disclosed herein are Bacillus coagulans strain RBE4-4, which is deposited at the China Center for Type Culture Collection (CCTCC) under accession number CCTCC M 2018310, and a method for producing lactic acid using such strain. The method comprises subjecting a fermentable sugar-containing substrate to a fermentation process with Bacillus coagulans strain RBE4-4.

### CROSS-REFERENCE TO RELATED APPLICATION

This application claims priority of Taiwanese Patent Application No. 107122573, filed on Jun. 29, 2018.

### FIELD

The present disclosure relates to lactic acid-producing Bacillus coagulans strain RBE4-4, which has been deposited at the Biosource Collection and Research Center (BCRC) of the Food Industry Research and Development Institute (FIRDI) under accession number BCRC 910831, as well as at the China Center for Type Culture Collection (CCTCC) under accession number CCTCC M 2018310. The present disclosure also relates to use of such strain to efficiently produce lactic acid.

### BACKGROUND

Cellulosic biomass is a renewable energy resource that can be massively produced from residues arising from industrial, agricultural, or forestry operation. Conversion of cellulosic biomass to lactic acid through a biological process has been widely investigated for further use.

When cellulosic biomass is applied for conducting fermentation with a microorganism to produce lactic acid, normally it is necessary to first subject the cellulosic biomass applied to a suitable saccharification process, so as to release fermentable sugars, including hexoses (mainly glucose) and pentoses (mainly xylose), from the cellulose and hemicelluloses in the cellulosic biomass applied. Accordingly, the substrate thus obtained, e.g. a cellulosic hydrolysate, can be fermented.

Since Bacillus coagulans can ferment pentoses and hexoses to produce lactic acid, and since the lactic acid thus produced is almost always L-form lactic acid and has an optical purity of nearly 100%, Bacillus coagulans has been widely used in producing lactic acid through fermentation of cellulosic biomass. Furthermore, Bacillus coagulans is acid-tolerant and heat-resistant and hence can conduct fermentation at a relatively low pH and a relatively high temperature, such that the risk of microbial contamination can be reduced, and such that sterilized operation and relevant sterilization processes can be further dispensed with (Qin J. et al. (2009), PLoS One, 4(2):e4359; Xue Z. W. et al. (2012), Springerplus., 1:43).

As reported in Patel M. A. et al. (2006), Appl. Environ. Microbial., 72(5):3228-35, 380 bacterial strains capable of utilizing xylose were isolated from soil. Among these strains, it was proved by experiments that Bacillus coagulans strains 17C5 and 36D1 are able to produce lactic acid from the fermentable sugars in a sugarcane bagasse hydrolysate treated with calcium hydroxide overliming, and have a desired lactic acid yield.

In addition, as described in Rhee M. S. et al. (2011), Stand. Genomic. Sci., 5(3):331-40, Bacillus coagulans strain 36D1 was further subjected to carbohydrate fermentation profile analysis, and the result indicated that Bacillus coagulans strain 36D1 can utilize glucose, xylose, arabinose, galactose, maltose, fructose, and cellobiose, but is unable to utilize cellulose and xylan.

Cellulosic biomass subjected to saccharification normally contains, in addition to fermentable sugars, fermentation inhibitors (for example, acetic acid, furfural, hydroxymethyl furfural (HMF), phenolic compounds, etc.) resulting from degradation of hemicellulose and fermentable sugars. Such inhibitors hinder the growth and fermentation performance of a microorganism, thus negatively affecting the lactic acid yield.

In order to overcome the adverse effect of fermentation inhibitors, numerous detoxification processes have been proposed, including: (1) physical detoxification, such as evaporation and membrane mediated detoxification; and (2) chemical detoxification, such as a treatment with calcium hydroxide overliming as described above, neutralization, an activated charcoal treatment, and a treatment with an ion exchange resin; and (3) biological detoxification, such as a treatment with laccase or lignin peroxidase. However, the aforesaid detoxification processes not only render the fermentation procedure complicated, but also increase the necessary cost. Besides, loss of fermentable sugars might occur during the aforesaid detoxification processes.

In view of the foregoing, the applicant has endeavored to develop a Bacillus coagulans strain that has excellent lactic acid-producing ability and that is highly resistant to fermentation inhibitors.

### SUMMARY

Accordingly, the present disclosure provides Bacillus coagulans strain RBE4-4, which is deposited at the China Center for Type Culture Collection (CCTCC) under accession number CCTCC M 2018310.

The present disclosure further provides a method for producing lactic acid, which comprises subjecting a fermentable sugar-containing substrate to a fermentation process with Bacillus coagulans strain RBE4-4 as described above.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described. For clarity, the following definitions are used herein.

The method for converting cellulosic biomass into lactic acid via a microorganism has been widely investigated. However, when cellulosic biomass is subjected to a pretreatment or a hydrolysis treatment, fermentation inhibitors (such as acetic acid, furfural, hydroxymethyl furfural, phenolic compounds, etc.) are often generated, thus adversely affecting the ability of a microorganism to produce lactic acid through fermentation. Accordingly, the applicant strived to develop a lactic acid bacteria strain that has excellent lactic acid-producing ability and that is highly tolerant to fermentation inhibitors.

The applicant obtained five lactic acid bacteria isolates from soil via isolation and screening processes, and evaluated their ability to ferment pentoses and hexoses so as to further select a lactic acid bacteria isolate that can co-ferment pentoses and hexoses to massively produce lactic acid. The selected lactic acid bacteria isolate was subjected to acclimatization using a cellulosic hydrolysate, such that lactic acid bacteria strain RBE4-4 having excellent tolerance to fermentation inhibitors was obtained. By virtue of characteristic analysis, lactic acid bacteria strain RBE4-4 was identified as a Bacillus coagulans strain. Therefore, lactic acid bacteria strain RBE4-4 is also referred to as Bacillus coagulans strain RBE4-4. Such strain has been deposited at the Biosource Collection and Research Center (BCRC) of the Food Industry Research and Development Institute (FIRDI) under accession number BCRC 910831 since Mar. 2, 2018, as well as at the China Center for Type Culture Collection (CCTCC) under accession number CCTCC M 2018310 in accordance with the Budapest Treaty since May 28, 2018.

Furthermore, the applicant used a cellulosic hydrolysate as a substrate to conduct separate hydrolysis and co-fermentation (SHCF), or simultaneous saccharification and co-fermentation (SSCF) by virtue of Bacillus coagulans strain RBE4-4, and verified that Bacillus coagulans strain RBE4-4 exhibits satisfactory lactic acid productivity in terms of any of these two fermentation processes.

Thus, the present disclosure provides Bacillus coagulans strain RBE4-4 as described above and a method for producing lactic acid using the same. The method of the present disclosure comprises subjecting a fermentable sugar-containing substrate to a fermentation process with Bacillus coagulans strain RBE4-4.

As used herein, the term. “fermentable sugar” refers to any carbohydrate (e.g. a monosaccharide, a disaccharide, and an oligosaccharide) that is water-soluble and can be used as a carbon source by Bacillus coagulans. The fermentable sugar-containing substrate contains at least one fermentable sugar, suitable examples of which include, but are not limited to, glucose, xylose, arabinose, fructose, galactose, cellobiose, mannose, rhamnose, maltose, lactose, melibiose, and trehalose. In certain embodiments, the at least one fermentable sugar in the fermentable sugar-containing substrate is selected from the group consisting of glucose, xylose, arabinose, mannose, cellobiose, galactose, and combinations thereof.

According to the present disclosure, the fermentable sugar-containing substrate is prepared from biomass using a saccharification process.

The aforesaid saccharification process for preparing the fermentable sugar-containing substrate may be terminated before the fermentation process, or may be still conducted during the fermentation process. When the saccharification process for preparing the fermentable sugar-containing substrate is to be terminated before the fermentation process, such process may be designed to substantially completely or partially hydrolyze the cellulose of the biomass before the fermentation process. When the saccharification process for preparing the fermentable sugar-containing substrate is to be still conducted during the fermentation process, such process may be designed to substantially completely or partially hydrolyze the remaining cellulose during the fermentation process.

According to the present disclosure, the fermentation process in the method of the present disclosure may be the SHCF process or the SSCF process. When the SHCF process is intended, the saccharification process for preparing the fermentable sugar-containing substrate may be conducted to substantially completely hydrolyze the cellulose in the biomass before the fermentation process, and hence may be terminated before the fermentation process. When the SSCF process is intended, the saccharification process for preparing the fermentable sugar-containing substrate may be conducted to partially hydrolyze the cellulose in the biomass before the fermentation process, and hence may be still conducted during the fermentation process to hydrolyze the cellulose remaining in the fermentable sugar-containing substrate.

As used herein, the terms “biomass”, “cellulosic biomass”, and “lignocellulosic biomass” are interchangeable, and refer to any cellulosic material that contains cellulose, hemicellulose, lignin, starch, an oligosaccharide, and/or a monosaccharide.

According to the present disclosure, the biomass may be derived from a single source, or may be a mixture derived from multiple sources. For instance, the biomass may be a mixture of corn stover and a corn cob, or a mixture of grass and a leaf.

Suitable examples of the biomass include, but are limited to, bioenergy crops, agricultural residues, municipal solid wastes, industrial solid wastes, sludge from paper manufacture, yard wastes, wood wastes, forestry wastes, and combinations thereof.

In certain embodiments, the biomass is selected from the group consisting of miscanthus, softwood, hardwood, a corn cob, crop residues (e.g. corn husks), corn stover, grass, wheat straw, barley straw, hay, rice straw, switchgrass, waste paper, sugarcane bagasse, a sorghum plant material, a soybean plant material, a ground material prepared from grains, trees, branches, roots, leaves, sawdust, shrubs and bushes, vegetables, fruits, flowers, and combinations thereof. In an exemplary embodiment, the biomass is rice straw.

As used herein, the terms “saccharification” and “hydrolysis” are interchangeable, and refer to generation of a fermentable sugar from a polysaccharide (such as cellulose, hemicellulose, etc.) in the biomass.

According to the present disclosure, the saccharification process for preparing the fermentable sugar-containing substrate may be an enzymatic hydrolysis process employing cellulase. The operation conditions of the enzymatic hydrolysis process employing cellulase are within the scope of expertise and routine skill of those skilled in the art. In addition to cellulase, the enzymatic hydrolysis process may further employ hemicellulase (i.e. a mixture of cellulase and hemicellulase may be used to conduct the enzymatic hydrolysis process).

In certain embodiments, the saccharification process, which employs the mixture of cellulase and hemicellulase, may be conducted at a temperature of 50° C. to 65° C. under stirring for 48 to 72 hours, so as to substantially completely hydrolyze the cellulose of the biomass before the fermentation process. In an exemplary embodiment, such saccharification process for substantially completely hydrolyzing the cellulose of the biomass before the fermentation process is conducted at a temperature of 50° C. to 55° C. under stirring for 48 hours, when the SHCF process is intended.

In other embodiments, the saccharification process, which employs the mixture of cellulase and hemicellulase, may be conducted at a temperature of 50° C. to 55° C. under stirring for 8 to 12 hours, so as to partially hydrolyze the cellulose of the biomass before the fermentation process, and hence may be still conducted during the fermentation process to hydrolyze the remaining cellulose. In an exemplary embodiment, such saccharification process for partially hydrolyzing the cellulose of the biomass before the fermentation process is conducted at a temperature of 50° C. to 55° C. under stirring for 12 hours, and is still conducted during the fermentation process also using the mixture of cellulase and hemicellulase to hydrolyze the remaining cellulose, when the SSCF process is intended.

According to the present disclosure, the fermentable sugar-containing substrate is a cellulosic hydrolysate containing glucose and xylose.

As used herein, the terms “cellulosic hydrolysate”, “lignocellulosic hydrolysate”, and “biomass hydrolysate” are interchangeable.

According to the present disclosure, the biomass may be subjected to a pretreatment before the saccharification process. The pretreatment may break down the structure of the lignin and cellulose in the biomass and/or facilitate hydrolysis of the hemicelluloses in the biomass, thereby enhancing the efficiency of subsequent saccharification. Suitable examples of the pretreatment include, but are not limited to, steam explosion, a thermal chemical pretreatment, mechanical disintegration, an acid treatment, organosolv, a sulfite pretreatment, and combinations thereof. In an exemplary embodiment, the pretreatment is acid-catalyzed steam explosion. The operation conditions of the pretreatment are within the expertise and routine skill of those skilled in the art.

According to the present disclosure, the fermentable sugar-containing substrate may further contain at least one fermentation inhibitor selected from the group consisting of acetic acid, furfural, hydroxymethyl furfural, and a phenolic compound.

The fermentable sugar-containing substrate may contain 1 g/L to 40 g/L of acetic acid. In certain embodiments, the fermentable sugar-containing substrate contains 1 g/L to 20 g/L of acetic acid.

The fermentable sugar-containing substrate may contain 0.5 g/L to 5 g/L of hydroxymethyl furfural. In certain embodiments, the fermentable sugar-containing substrate contains 1 g/L to 3 g/L of hydroxymethyl furfural.

The fermentable sugar-containing substrate may contain 0.5 g/L to 5 g/L of furfural. In certain embodiments, the fermentable sugar-containing substrate contains 1 g/L to 3 g/L of furfural.

The fermentable sugar-containing substrate may contain 0.3 g/L to 4 g/L of the phenolic compound. In certain embodiments, the fermentable sugar-containing substrate contains 0.5 g/L to 2.5 g/L of the phenolic compound.

According to the present disclosure, the fermentation process may be conducted at a pH ranging from 5 to 8. In certain embodiments, the fermentation process is conducted at a pH ranging from 5.5 to 7.0.

The disclosure will be further described by way of the following examples. However, it should be understood that the following examples are solely intended for the purpose of illustration and should not be construed as limiting the disclosure in practice.

### Examples

### General Experimental Procedures:

### 1. High Performance Liquid Chromatography (HPLC)

In the following experiments, the concentration (g/L) of glucose, xylose, lactic acid, acetic acid, hydroxymethyl furfural, and/or furfural in cellulosic hydrolysates and fermentation cultures was determined using an HPLC system (Dionex Ultimate 3000) equipped with a refractive index (RI) detector according to the laboratory analytical procedures (LAPs) for standard biomass analysis provided by the National Renewable Energy Laboratory (NREL) of the United States. The operating conditions of HPLC are shown in Table 1 below.

TABLE 1
Operating conditions of HPLC

| | |
|---|---|
| Type of Column | AMINEX ® HPX-87H |
| | (BioRad, Cat No. 1250140) |
| Column size | 300 mm × 7.8 mm |
| Column temperature | 65° C. |
| Temperature of RI | 45° C. |
| detector | |
| Mobile phase | 5 mM sulfuric acid solution |
| Flow rate | 0.6 mL/min |

### Example 1. Screening of Lactic Acid Bacteria Isolates Having Lactic Acid-Producing Ability and Able to Co-Ferment Pentose and Hexose

### A. Isolation and Screening of Lactic Acid Bacteria Having Lactic Acid-Producing Ability

Soil collected from Yangmingshan National Park (Taipei, Taiwan) served as the source for lactic acid bacteria. Isolation, screening, and purification of lactic acid bacteria having lactic acid-producing ability were conducted as follows. 2 g of the soil was added into 50 mL of a liquid medium containing 10 g/L yeast extract and 20 g/L xylose, followed by mixing evenly. Cultivation was conducted in a thermostatic shaking incubator (50° C., 150 rpm) for 12 to 16 hours. The resulting culture was subjected to 10-fold serial dilution using sterile water, so as to obtain diluted cultures prepared by different dilution factors (10¹ to 10⁷). 0.1 mL of a respective diluted culture was evenly spread onto a first calcium carbonate agar plate (containing 10 g/L yeast extract, 20 g/L xylose, 5 g/L calcium carbonate, and 15 g/L agar; having a pH of 6), followed by being left standing for cultivation to be conducted at 50° C. for 24 to 48 hours. Based on the fact that lactic acid produced by lactic acid bacteria can react with calcium carbonate to form colorless calcium lactate, colonies able to rapidly grow on the first calcium carbonate agar plate and form a transparent circle were selected. The four-quadrant streak method was applied to respectively spread the selected colonies onto four quadrants of a second calcium carbonate agar plate (containing 10 g/L yeast extract, 50 g/L xylose, 15 g/L calcium carbonate, and 15 g/L agar; having a pH of 6), followed by being left standing for cultivation to be conducted at 50° C. for 24 to 48 hours. Large colonies having a large transparent circle were selected, and were subjected to screening and purification several times using a new second calcium carbonate agar plate via the procedure described above. The five lactic acid bacteria isolates thus obtained are referred to as RBE1, RBE2, RBE3, RBE4, and RBE5, respectively.

### B. Preparation of Inoculums of Lactic Acid Bacteria Isolates RBE1 to RBE5

A respective one of lactic acid bacteria isolates RBE1 to RBE5 obtained in section A of this example was inoculated at 5×10⁹ cells/mL into 90 mL of YPD40 medium (containing 10 g/L of yeast extract, 40 g/L of glucose, and 20 g/L of peptone), followed by adding a suitable amount of CaCO₃ to adjust the pH value to 5 to 7. Cultivation was conducted in a thermostatic shaking incubator (50° C., 150 rpm) for 24 hours. The resulting culture was used as an inoculum of the respective lactic acid bacteria isolate in the experiment below.

### C. Screening of Lactic Acid Bacteria Isolates Able to Co-Ferment Pentose and Hexose to Produce Lactic Acid

The inoculums of lactic acid bacteria isolates RBE1 to RBE5 obtained in section B of this example were each divided into a pentose group, a hexose group, and a dual sugar group. A respective one of the pentose, hexose, and dual sugar groups was inoculated at 10% (v/v) into 90 mL of a corresponding fermentation medium prepared according to the recipe shown in Table 2 below.

TABLE 2
Recipe of fermentation medium for respective
group of inoculum

| | | | Calcium | Yeast |
|---|---|---|---|---|
| | Glucose | Xylose | carbonate | extract |
| Group | (g/L) | (g/L) | (g/L) | (g/L) |
| Pentose | — | 50 | 30 | 10 |
| Hexose | 100 | — | 60 | 10 |
| Dual sugar | 85 | 45 | 75 | 10 |

The pentose, hexose, and dual sugar groups were allowed to conduct a fermentation reaction under an anaerobic condition in a thermostatic shaking incubator (50° C. to 52° C., 150 rpm) respectively for 16, 24, and 54 hours. Subsequently, the resulting fermentation culture of the respective group was subjected to centrifugation at 12,000 rpm for 6 minutes. The supernatant thus obtained (i.e. a fermentation product) was subjected to determination of lactic acid content according to the method described in section 1 of General Experimental Procedures.

The lactic acid productivity was calculated by substituting the lactic acid content determined and the fermentation time into the following Equation (1):

A=B/C (1)
A==lactic acid productivity (g/L/h)
B==lactic acid content determined (g/L)
C=fermentation time (h)
   The result is shown in Table 3 below.

TABLE 3
Lactic acid productivity of three groups of
inoculum prepared from respective lactic
acid bacteria isolate

| Lactic acid | | | | | |
|---|---|---|---|---|---|
| bacteria | Lactic acid productivity (g/L/h) | | | | |
| isolate | RBE1 | RBE2 | RBE3 | RBE4 | RBE5 |
| Pentose group | 1.04 | 2.1 | 1.23 | 2.837 | 2.3 |
| Hexose group | 3.2 | 3 | 2.4 | 3.95 | 2.8 |
| Dual sugar | 1.25 | 2.2 | 1.32 | 2.314 | 2 |
| group | | | | | |

As shown in Table 3, among all the lactic acid bacteria isolates, lactic acid bacteria isolate RBE4 had the highest lactic acid productivity in terms of any one of the pentose, hexose, and dual sugar groups. Therefore, the applicant opined that lactic acid bacteria isolate RBE4, compared to the other lactic acid bacteria isolates obtained, has the most potential for efficiently producing lactic acid, and conducted acclimatization on lactic acid bacteria isolate RBE4 as follows.

### Example 2. Acclimatization of Lactic Acid Bacteria Isolate RBE4 with Cellulosic Hydrolysate

In this example, lactic acid bacteria isolate RBE4 was subjected to acclimatization using a cellulosic hydrolysate, so as to obtain a lactic acid bacteria strain which has improved tolerance to the fermentation inhibitors in such cellulosic hydrolysate and hence has enhanced lactic acid productivity.

### Experimental Materials:

### 1. Cellulosic Hydrolysate

Rice straw (purchased from Hong Yuan Agricultural Production Company), which served as cellulosic biomass, was cut into a length of 0.5 cm, followed by crushing with a crusher. 30 g/L of a sulfuric acid solution was evenly mixed with the resulting crushed rice straw, and the mixture thus obtained was left standing at 121° C. for 120 to 180 minutes for acid impregnation. Subsequently, the mixture was placed in a vertical cyrlindrical-shaped high-pressure digester tank (Lucky Seven Industrial Co., Ltd.), and steam was introduced. Heating was conducted at a temperature of 190° C. to 200° C. for 5 minutes. The pretreatment product resulting from the aforesaid acid-catalyzed steam explosion pretreatment was subjected to high-pressure filtration using a plate and frame filter press (Model No. FP500-5, Water Power Technology Corp.), so as to adjust the solid content of the pretreatment product to about 25%. Subsequently, a 25% ammonia solution was used to adjust the pH value to 4.8 to 5.5. An enzyme blend (Novozymes Cellic® CTec3) composed of cellulase and hemicellulase was added at an enzyme loading ranging from 15 to 30 FPU/g of cellulosic biomass, so that a cellulolytic process was conducted at a temperature of 50° C. to 55° C. and a stirring speed of 70 rpm for 48 hours. Afterwards, high-pressure filtration was performed to remove the solid formed, such that a cellulosic hydrolysate was obtained.

The contents of carbohydrates and fermentation inhibitors in the cellulosic hydrolysate were determined according to the method described in section 1 of General Experimental Procedures. The result is shown in Table 4 below.

TABLE 4
Contents of carbohydrates and fermentation
inhibitors in cellulosic hydrolysate

| Component | Content (g/L) |
|---|---|
| Glucose | 97 |
| Xylose | 17 |
| Acetic acid | 3-5 |
| Furfural | 2-3.5 |
| Hydroxymethyl | 1-2 |
| furfural | |

Lastly, 3 g/L of yeast extract was added to the cellulosic hydrolysate, followed by sterilization at 121° C. for 20 minutes. Therefore, a sterile cellulosic hydrolysate was obtained.

### Experimental Procedures:

The inoculum of lactic acid bacteria isolate RBE4 obtained in section B of Example 1 was inoculated at 1% (v/v) into 99 mL of the sterile cellulosic hydrolysate, followed by cultivation under an anaerobic condition in a thermostatic shaking incubator (50° C. to 52° C., 150 rpm) for 72 hours.

The resulting culture was spread onto a cultivation plate (containing 10 g/L of yeast extract, 50 g/L of xylose, 15 g/L of calcium carbonate, and 15 g/L of agar; having a pH of 6), followed by cultivation at 50° C. for 24 to 48 hours. Fast-growing colonies were selected. The aforesaid inoculation, cultivaton, and selection steps were repeated 100 times, such that an acclimatized lactic acid bacteria strain, referred to as RBE4-4, was obtained.

### Example 3. Evaluation for Tolerance of Lactic Acid Bacteria Strain RBE4-4 to Fermentation Inhibitors

The tolerance of lactic acid bacteria strain RBE4-4 to fermentation inhibitors (including furfural, hydroxymethyl furfural, and acetic acid) was evaluated generally according to the method described in CN 103667110 A.

First, the tolerance to furfural was analyzed. Specifically, lactic acid bacteria strain RBE4-4 was inolculated at 5×10⁹ cells/mL into 90 mL of YPD40 medium, followed by overnight cultivation in a thermostatic shaking incubator (50° C., 150 rpm). Subsequently, the resulting culture of lactic acid bacteria strain RBE4-4 was divided into a control group and 5 experimental groups (i.e. Experimental Groups 1 to 5). A respective one of Experimental Groups 1 to 5 was inoculated at 1% (v/v) into 99 mL of Difco™ Lactobacilli MRS broth (BD Bioscience) supplemented with a corresponding concentration (i.e. 1 to 5 g/L) of furfural. The control group was inoculated at 1% (v/v) into 99 mL of Difco™ Lactobacilli MRS broth supplemented with no furfural.

Cultivation was conducted in a thermostatic shaking incubator (50° C., 150 rpm) for 24 hours. Subsequently, 100 μL of the resulting culture of the respective group was added into a 96-well plate. The absorbance at 420 nm (0D420) was determined using a spectrophotometer (Thermo Scientific, BioMate™ 3S).

The relative optical density (ROD) (%) of the respective group was calculated by substituting the OD₄₂₀ determined into the following Equation (2).

D=(E/F)×100 (2)
D=ROD (%)
E=OD₄₂₀ of respective group
F=OD₄₂₀ of control group

The tolerance of lactic acid bacteria strain RBE4-4 to hydroxymethyl furfural and acetic acid was evaluated generally according to the aforesaid procedure for evaluating tolerance to furfural, except that a respective one of hydroxymethyl furfural (1 to 5 g/L) and acetic acid (10 to 40 g/L) was used instead of furfural.

In addition, for the sake of comparison, lactic acid bacteria isolate RBE4, which was never acclimatized, was subjected to the same experiment.

The results regarding the tolerance of lactic acid bacteria isolate RBE4 and lactic acid bacteria strain RBE4-4 to different fermentation inhibitors are respectively shown in Tables 5 to 7 below.

TABLE 5
ROD determined under different concentrations of furfural

ROD (%)

| | | Lactic | Lactic |
|---|---|---|---|
| | | acid | acid |
| | Furfural | bacteria | bacteria |
| | concentration | isolate | strain |
| Group | (g/L) | RBE4 | RBE4-4 |
| Control group | 0 | 100 | 100 |
| Experimental | 1 | 96.8 | 100 |
| Group 1 | | | |
| Experimental | 2 | 96.3 | 98 |
| Group 2 | | | |
| Experimental | 3 | 30.3 | 96 |
| Group 3 | | | |
| Experimental | 4 | 0 | 50.7 |
| Group 4 | | | |
| Experimental | 5 | 0 | 44.1 |
| Group 5 | | | |

TABLE 6
ROD determined under different concentrations
of hydroxymethyl furfural

ROD (%)

| | | Lactic | Lactic |
|---|---|---|---|
| | Hydroxymethyl | acid | acid |
| | furfural | bacteria | bacteria |
| | concentration | isolate | strain |
| Group | (g/L) | RBE4 | RBE4-4 |
| Control | 0 | 100 | 100 |
| group | | | |
| Experimental | 1 | 100 | 100 |
| Group 1 | | | |
| Experimental | 2 | 92.4 | 100 |
| Group 2 | | | |
| Experimental | 3 | 15.3 | 96.7 |
| Group 3 | | | |
| Experimental | 4 | 0 | 77.2 |
| Group 4 | | | |
| Experimental | 5 | 0 | 30.7 |
| Group 5 | | | |

TABLE 7
ROD determined under different concentrations of acetic acid

ROD (%)

| | | Lactic | Lactic |
|---|---|---|---|
| | | acid | acid |
| | Acetic acid | bacteria | bacteria |
| | concentration | isolate | strain |
| Group | (g/L) | RBE4 | RBE4-4 |
| Control | 0 | 100 | 100 |
| group | | | |
| Experimental | 10 | 100 | 100 |
| Group 1 | | | |
| Experimental | 20 | 76.5 | 99.5 |
| Group 2 | | | |
| Experimental | 30 | 36.86 | 80.7 |
| Group 3 | | | |
| Experimental | 35 | 0 | 76.3 |
| Group 4 | | | |
| Experimental | 40 | 0 | 36 |
| Group 5 | | | |

As shown in Table 5, in the presence of furfural at any of the five concentrations tested, the ROD of lactic acid bacteria strain RBE4-4 was higher than that of lactic acid bacteria isolate RBE4, indicating that lactic acid bacteria strain RBE4-4 is more viable than lactic acid bacteria isolate RBE4 in the presence of furfural. In particular, when the furfural concentration was 4 g/L or higher, lactic acid bacteria isolate RBE4 was not viable, whereas lactic acid bacteria strain RBE4-4 was viable. Moreover, the viability of lactic acid bacteria strain RBE4-4 under 5 g/L of furfural was even significantly better than that of lactic acid bacteria isolate RBE4 under 3 g/L of furfural.

Referring to Table 6, when the hydroxymethyl furfural concentration was 2 g/L or higher, the ROD of lactic acid bacteria strain RBE4-4 was higher than that of lactic acid bacteria isolate RBE4, manifesting that lactic acid bacteria strain RBE4-4 is generally more viable than lactic acid bacteria isolate RBE4 in the presence of hydroxymethyl furfural. Particularly, when the hydroxymethyl furfural concentration was 4 g/L or higher, lactic acid bacteria isolate RBE4 was not viable, whereas lactic acid bacteria strain RBE4-4 was viable. Furthermore, the viability of lactic acid bacteria strain RBE4-4 under 5 g/L of hydroxymethyl furfural was even significantly better than that of lactic acid bacteria isolate RBE4 under 3 g/L of hydroxymethyl furfural.

As shown in Table 7, when the acetic acid concentration was 20 g/L or higher, the ROD of lactic acid bacteria strain RBE4-4 was higher than that of lactic acid bacteria isolate RBE4, indicating that lactic acid bacteria strain RBE4-4 is generally more viable than lactic acid bacteria isolate RBE4 in the presence of acetic acid. In particular, when the acetic acid concentration was 35 g/L or higher, lactic acid bacteria isolate RBE4 was not viable, whereas lactic acid bacteria strain RBE4-4 was viable. Moreover, the viability of lactic acid bacteria strain RBE4-4 under 40 g/L of acetic acid was even similar to that of lactic acid bacteria isolate RBE4 under 30 g/L of acetic acid.

In view of the aforesaid experimental results, it can be concluded that lactic acid bacteria strain RBE4-4, which is a lactic acid bacteria strain prepared by acclimatizing lactic acid bacteria isolate RBE4 with a cellulosic hydrolysate, has stronger tolerance to fermentation inhibitors (including furfural, hydroxymethyl furfural, and acetic acid) compared to the parent strain, i.e., lactic acid bacteria isolate RBE4. Thus, lactic acid bacteria strain RBE4-4 is not a naturally occurring lactic acid bacterium like lactic acid bacteria isolate RBE4.

### Example 4. Characteristic Analysis of Lactic Acid Bacteria Strain RBE4-4

In order to identify the bacterial species of lactic acid bacteria strain RBE4-4, the following preliminary characteristic determination, determination of carbohydrate fermentation profiling, 16S rDNA sequencing, and fermentation tests with difference carbon sources were conducted.

### A. Preliminary Characteristic Determination

The morphopholgy and antibiotic resistance of lactic acid bacteria strain RBE4-4 were determined using techniques well-known in the art.

The results of the aforesaid preliminary characteristic determination indicate that lactic acid bacteria strain RBE4-4 is a Bacillus species, and has no resistance to ampicillin, chloramphenicol, kanamycin, and tetracycline.

### B. Carbohydrate Fermentation Profiling

The carbohydrate fermentation profile of lactic acid bacteria strain RBE4-4 was determined by the Bioresource Collection and Research Center (BCRC) of the Food Industry Research and Development Institute (FIRDI) using API 50 CHB Identification Kit V4.1 (bioMérieux). The result is shown in Table 8 below.

TABLE 8
Carbohydrate fermentation profile of lactic
acid bacteria strain RBE4-4

| | Capability of fermenting |
|---|---|
| | carbohydrate tested to |
| Carbohydrate | produce acid |
| Glycerol | + |
| Erythritol | + |
| D-Arabinose | − |
| L-Arabinose | + |
| Ribose | + |
| D-Xylose | + |
| L-Xylose | − |
| Adonitol | − |
| Methyl-β-D-xylopyranoside | − |
| Galactose | + |
| Glucose | + |
| Fructose | + |
| Mannose | + |
| Sorbose | − |
| Rhamnose | + |
| Dulcitol | − |
| Inositol | − |
| Mannitol | − |
| Sorbitol | + |
| Methyl-α-D-mannopyranoside | − |
| Methyl-α-D-glucopyranoside | − |
| N-acetylglucosamine | + |
| Amygdalin | − |
| Arbutin | + |
| Esculin | + |
| Salicin | + |
| Cellobiose | + |
| Maltose | + |
| Lactose | + |
| Melibiose | + |
| D-Saccharose | − |
| Trehalose | + |
| Inulin | − |
| Melezitose | − |
| Raffinose | − |
| Amidon | − |
| Glycogen | − |
| Xylitol | − |
| Gentiobiose | − |
| D-Turanose | − |
| D-Lyxose | − |
| Tagatose | − |
| D-Fucose | − |
| L-Fucose | − |
| D-Arabitol | − |
| L-Arabitol | − |
| Gluconate | − |
| Potassium 2-ketogluconate | − |
| Potassium 5-ketogluconate | − |

*: “+” indicates that lactic acid bacteria strain RBE4-4 is capable of fermenting the carbohydrate tested to produce an acid, whereas “−” indicates that the strain has no such capability.

The aforesaid result was subjected to comparison with the data in the APIWEB™ on-line bacteria and yeast database, and it was found that the carbohydrate fermentation profile of lactic acid bacteria strain RBE4-4 of the present disclosure has 95.5% identity to that of Bacillus coagulans.

C.16S rDNA Sequencing

Bacterial Genomic DNA Purification Kit (Scientific Biotech Corp.) was used to extract the genomic DNA of lactic acid bacteria strain RBE4-4. Subsequently, polymerase chain reaction (PCR) was conducted using the genomic DNA of lactic acid bacteria strain RBE4-4 serving as a template, as well as a forward primer 27F (SEQ ID NO: 1) and a reverse primer pH′ (SEQ ID NO: 2) designed for the 16S rDNA of bacteria respectively according to Weisburg W. G. et al. (1991), J. Bacteriol., 173 (2): 697-703 and Edwards U. et al. (1989), Nucleic Acids Res., 17(19): 7843-53. Therefore, the 16S rDNA fragments of lactic acid bacteria strain RBE4-4 were amplified.

Sequencing was conducted by Mission Biotech Co. Ltd., such that the 16S rDNA sequence (SEQ ID NO: 3) of lactic acid bacteria strain RBE4-4 was obtained.

Through comparison with the data in the NCBI's gene database, it was found that the 16S rDNA sequence of lactic acid bacteria strain RBE4-4 has 99% identity to a part of the 16S rDNA sequence (Genbank accession number CP003056.1) of Bacillus coagulans strain 36D1.

In view of the aforesaid experimental results obtained in sections A to C of this example, lactic acid bacteria strain RBE4-4 of the present disclosure is identified as Bacillus coagulans. In order to confirm whether Bacillus coagulans strain RBE4-4 (i.e. lactic acid bacteria strain RBE4-4) is a novel Bacillus coagulans strain, the following experiment was conducted.

D. Fermentation Tests with Different Carbon Sources

According to the description in Rhee M. S. et al. (2011), supra, Bacillus coagulans strain 36D1 can use glucose as a carbon source for growth or fermentation, but is unable to utilize cellulose and xylan. Thus, Bacillus coagulans strain RBE4-4 was subjected to fermentation tests using glucose, cellulose and xylan as carbon sources.

An inoculum of lactic acid bacteria strain RBE4-4 was prepared according to the procedure described in section B of Example 1. Afterwards, the inoculum of lactic acid bacteria strain RBE4-4 was divided into a glucose group, a cellulose group, and a xylan group. The respective group was inoculated at 10% (v/v) into 90 mL of a liquid culture medium containing 20 g/L of a corresponding carbon source (glucose, cellulose or xylan) and 10 g/L of yeast extract.

Fermentation was allowed to proceed under an anaerobic condition in a thermostatic shaking incubator (50° C., 150 rpm) for 48 hours. 100 μL of the resulting culture of the respective group was added into a 96-well plate. Subsequently, the absorbance at 420 nm (0D420) was determined using a spectrophotometer. The result is shown in Table 9 below.

TABLE 9
OD₄₂₀ under different carbon sources

| Group | OD₄₂₀ |
|---|---|
| Glucose group | 5.93 |
| Cellulose group | 9.18 |
| Xylan group | 23.6 |

As shown in Table 9, lactic acid bacteria strain RBE4-4 is able to utilize glucose, cellulose and xylan as carbon sources for growth. Particularly, when xylan, instead of glucose and cellulose, served as a carbon source, lactic acid bacteria strain RBE4-4 had the highest growth rate.

In view of the aforementioned experimental result, the applicant deems that lactic acid bacteria strain RBE4-4 of the present disclosure is a novel Bacillus coagulans strain.

Bacillus coagulans strain RBE4-4 of the present disclosure has been deposited at the BCRC of the FIRDI (331 Shih-Pin Rd., Hsinchu City 300, Taiwan) under accession number BCRC 910831 since Mar. 2, 2018, and at the China Center for Type Culture Collection (CCTCC) (Wuhan University, Wuhan, 430072, People's Republic of China) under accession number CCTCC M 2018310 since May 28, 2018.

### Example 5. Use of Cellulosic Hydrolysate as Substrate for Lactic Acid Fermentation

In order to investigate the ability of Bacillus coagulans strain RBE4-4 of the present disclosure to use a cellulosic hydrolysate as a substrate for lactic acid fermentation, the following experiment was performed. In addition, for the sake of comparison, lactic acid bacteria isolate RBE4 and Bacillus coagulans strain DSM1 (purchased from the BCRC of the FIRDI; deposited under accession number BCRC 10606 at the BCRC of the FIRDI and under accession number ATCC 7050 at the American Type Culture Collection) were subjected to the same experiment.

### A. Separate Hydrolysis and Co-Fermentation (SHCF)

10 g/L of yeast extract was added into a cellulosic hydrolysate as described in Experimental Materials of Example 2, which contained carbohydrates and fermentation inhibitors as shown in Table 4, followed by adding a suitable amount of a 25% ammonia solution to adjust the pH value to 5.5 to 7.0.

Afterwards, inoculums of lactic acid bacteria isolate RBE4, Bacillus coagulans strain RBE4-4, and Bacillus coagulans strain DSM1 were prepared according to the procedure described in section B of Example 1. A respective one of the three inoculums was inoculated at 10% (v/v) into 90 mL of the cellulosic hydrolysate. Fermentation was allowed to proceed under an anaerobic condition in a thermostatic shaking incubator (50° C., 150 rpm) for 48 hours. At 6 hour, 12 hour, and 24 hour after the beginning of fermentation, a suitable amount of a 25% ammonia solution was added to the respective culture so as to maintain the pH value at 5.5 to 7.0.

The resulting fermentation culture was subjected to centrifugation at 12,000 rpm for 6 minutes. The supernatant thus obtained (i.e. a fermentation product) was subjected to determination of lactic acid content according to the method described in section 1 of General Experimental Procedures.

The aforesaid experiment was repeated thrice. The experimental data are expressed as mean±SEM (standard error of the mean) and are shown in Table 10 below.

TABLE 10
Lactic acid productivity of different test strains

| | Lactic acid productivity |
|---|---|
| Test strain | (g/L/h) |
| DSM1 | 0.697 ± 0.2 |
| RBE4 | 1.69 ± 0.18 |
| RBE4-4 | 2.11 ± 0.14 |

As shown in Table 10, when the SHCF process was applied to ferment the cellulosic hydrolysate so as to produce lactic acid, the lactic acid productivity of Bacillus coagulans strain RBE4-4 was apparently higher than that of any of test strains RBE4 and DSM1.

### B. Simultaneous Saccharification and Co-Fermentation (SSCF)

A cellulosic hydrolysate was prepared in a manner similar to that for preparing the cellulosic hydrolysate described in Experimental Materials of Example 2, except that the cellulolytic process was conducted at a temperature of 50° C. to 55° C. and a stirring speed of 150 rpm for 12 hours to only partially hydrolyze the cellulose, and that the solid formed was not removed.

The contents of carbohydrates and fermentation inhibitors in the cellulosic hydrolysate were determined according to the method described in section 1 of General Experimental Procedures. The result is shown in Table 11 below.

TABLE 11
Contents of carbohydrates and fermentation
inhibitors in cellulosic hydrolysate
prepared from rice straw through partial
decomposition process

| Component | Content (g/L) |
|---|---|
| Glucose | 80.5 |
| Xylose | 27 |
| Acetic acid | 2.5 |
| Furfural | 2 |
| Hydroxymethyl | 1.2 |
| furfural | |

Subsequently, fermentation and determination of lactic acid content were conducted generally according to the procedure described in section A of this example, except that saccharication and fermentation were allowed to simultaneously proceed for 20 hours, and that at 6 hour and 12 hour after the beginning of fermentation, a suitable amount of a 25% ammonia solution was added to the respective culture so as to maintain the pH value at 5.5 to 7.0.

The result is shown in Table 12 below.

TABLE 12
Lactic acid productivity of different test strains

| | Lactic acid productivity |
|---|---|
| Test strain | (g/L/h) |
| DSM1 | 1.92 ± 0.48 |
| RBE4 | 3.91 ± 0.78 |
| RBE4-4 | 4.95 ± 0.825 |

As shown in Table 12, when the SSCF process was applied to ferment the cellulosic hydrolysate so as to produce lactic acid, the lactic acid productivity of Bacillus coagulans strain RBE4-4 was apparently higher than that of any of the test strains RBE4 and DSM1.

In view of the experimental results of this example, it can be verified that Bacillus coagulans strain RBE4-4 of the present disclosure is a novel and non-obvious Bacillus coagulans strain which has satisfactory ability to perform lactic acid fermentation, no matter which fermentation process (i.e. SHCF or SSCF) is conducted using a cellulosic hydrolysate as a substrate.

All patents and references cited in this specification are incorporated herein in their entirety as reference. Where there is conflict, the descriptions in this case, including the definitions, shall prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. Bacillus coagulans strain RBE4-4, which is deposited at the China Center for Type Culture Collection (CCTCC) under accession number CCTCC M 2018310.

2. A method for producing lactic acid, comprising:
subjecting a fermentable sugar-containing substrate to a fermentation process with Bacillus coagulans strain RBE4-4 as claimed in claim 1.

3. The method according to claim 2, wherein the fermentable sugar-containing substrate is prepared from biomass using a saccharification process.

4. The method according to claim 3, wherein the saccharification process is terminated before the fermentation process.

5. The method according to claim 4, wherein the saccharification process is conducted at a temperature of 50° C. to 55° C. under stirring.

6. The method according to claim 3, wherein the saccharification process is still conducted during the fermentation process.

7. The method according to claim 6, wherein the saccharification process is conducted at a temperature of 50° C. to 55° C. under stirring.

8. The method according to claim 3, wherein the fermentable sugar-containing substrate contains a fermentation inhibitor selected from the group consisting of acetic acid, furfural, hydroxymethyl furfural, a phenolic compound, and combinations thereof.

9. The method according to claim 8, wherein the fermentable sugar-containing substrate contains 1 g/L to 20 g/L of acetic acid.

10. The method according to claim 8, wherein the fermentable sugar-containing substrate contains 0.5 g/L to 5 g/L of hydroxymethyl furfural.

11. The method according to claim 8, wherein the fermentable sugar-containing substrate contains 0.5 g/L to 5 g/L of furfural.

12. The method according to claim 8, wherein the fermentable sugar-containing substrate contains 0.3 g/L to 4 g/L of the phenolic compound.

13. The method according to claim 2, wherein the fermentable sugar-containing substrate contains at least one fermentable sugar selected from the group consisting of glucose, xylose, arabinose, mannose, cellobiose, galactose, and combinations thereof.

14. The method according to claim 2, wherein the fermentation process is conducted at a pH ranging from 5.5 to 7.0.
